(19) United States
(12) Patent Application Publication
(10) Pub. No.: US 2011/0020302 A1
(43) Pub. Date: Jan. 27, 2011

(54) NATURAL PRESERVATIVE BLEND

(75) Inventors: DANIEL BANOV, SUGAR LAND, TX (US); AUGUST S. BASSANI, KATY, TX (US); CHRISTINE M. NGUYEN, HOUSTON, TX (US); FABIANA CAMPANATI VIEIRA BANOV, SUGAR LAND, TX (US)

Correspondence Address: SHOOK, HARDY & BACON LLP;INTELLECTUAL PROPERTY DEPARTMENT, 2555 GRAND BLVD, KANSAS CITY, MO, 64108-2613, US

(73) Assignee: PROFESSIONAL COMPOUNDING CENTERS OF AMERICA, LTD., HOUSTON, TX (US)

(21) Appl. No.: 12/508,302

(22) Filed: Jul. 23, 2009

(51) Int. Cl. A61K 35/74

(52) U.S. Cl. 424/93.44

(57) ABSTRACT

A composition to be used as a natural preservative is provided. The composition comprises 1.0 to 20% w/w one or more naturally occurring substances containing salicylate, 0.1 to 20% w/w one or more species of lonicera, 0.1 to 80% w/w one or more leuconostoc, and 0.1 to 10% w/w gluconic acid. The composition has antimicrobial properties and is in a liquid form.

### CROSS-REFERENCE TO RELATED APPLICATIONS

Not applicable.

### STATEMENT REGARDING FEDERALLY SPONSORED RESEARCH OR DEVELOPMENT

Not applicable.

### BACKGROUND

Traditional preservatives used for cosmetics, pharmaceuticals, food, and the like, are under increasing regulatory pressure and public concern over their safety and environmental impact. For instance, many preservatives are made from synthetic compounds, whose safety is under increased scrutiny, thus leading many to prefer more natural preservative alternatives. Preservation is important in many industries, such as the cosmetic and pharmaceutical industries, as the shelf life of many cosmetic and pharmaceutical products is significant. Further, many preservative formulations that are used today have an impact on the feel, texture, color, and smell of the product in which the preservative is used.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter. Embodiments of the present invention are directed to natural preservative blends, where a formulation is used as a preservative for cosmetic, pharmaceutical, in addition to other products.

In one embodiment, a composition to be used as a natural preservative is provided. The composition comprises 1.0 to 20% w/w one or more naturally occurring substances containing salicylate, 0.1 to 20% w/w one or more species of lonicera, 0.1 to 80% w/w one or more leuconostoc, and 0.1 to 10% w/w gluconic acid.

In another embodiment, a composition to be used as a natural preservative is provided. The composition comprises a combination of populus tremuloides bark extract, lonicera japonica, leuconostoc filtrate and glucono-delta-lactone.

In yet another embodiment, a method for topically applying a natural preservative blend to a skin surface is provided. A natural preservative blend comprising one or more naturally occurring substances containing salicylate, one or more species of lonicera, one or more leuconostoc, and gluconic acid is provided. The components of the natural preservative blend are provided in an amount sufficient to exhibit antimicrobial activity. The natural preservative blend is contacted with the skin surface. The natural preservative blend is applied to the skin surface such that the natural preservative blend exhibits antimicrobial activity.

Additional aspects of the invention, together with the advantages and novel features appurtenant thereto, will be set forth in part in the description that follows, and in part will become apparent to those skilled in the art upon examination of the following, or may be learned from the practice of the invention. The objects and advantages of the invention may be realized and attained by means, instrumentalities, and combinations particular pointed out in the appended claims.

### DETAILED DESCRIPTION

Embodiments of the present invention are directed to a natural preservative composition or formulation containing one or more naturally occurring substances containing salicylate, one or more species of lonicera, and gluconic acid. The composition may be used as a preservative for various products, including cosmetics and pharmaceuticals. While the composition is safe and effective, it is comprised of natural ingredients that provide antimicrobial protection of various topical formulations.

The natural preservative blend described herein may be used as a natural alternative to synthetic preservatives, but may also be used, in one embodiment, as a topical antimicrobial that addresses problem skin or specific scalp conditions. The composition may be easily incorporated into a variety of products that may need to be preserved. For instance, the composition, in one embodiment, is a liquid and is miscible with many organic solvents, surfactants, and emulsifiers. As the composition may be chemically inert, it is compatible with the majority of types of chemical compounds. The composition is also non-volatile, and thus there is no loss of preservative activity from the product, even after prolonged exposure to air or in storage. In one embodiment, the formulation is paraben-free. In another embodiment, the formulation does not contain formaldehyde and is not a formaldehyde-donor. In another embodiment, formulation does not contain iodine and is not an iodine-donor.

While there is no official legal definition of the term “natural” as it relates to cosmetic ingredients, Ecocert, BDIH (the Federation of German Industries and Trading) and the Soil Association both agree that the term implies a ban on the use of petrochemical derived ingredients, silicones, ethoxylated raw materials, and halogen organic compounds.

Further, the combination of the compounds discussed herein allows for each component to be used in smaller than usual doses, thus limiting any potential effects of the compounds. While each component may be used in small amounts, the combination of the above-mentioned components allows for a strong and effective compound that maintains its antimicrobial activities in the presence of materials such as proteins, gums, and ionic solutions, and maintains its antimicrobial activities in acidic, neutral, and even alkaline pH conditions.

While many preservative blends are known to be odorous and may change the color of the product with which the preservative blend is incorporated, the composition described herein does not cause any changes in color or odor to the final product. The formulation, in one embodiment, is fully biodegradable at the extremely dilute conditions as found in the effluents, and thus presents no pollution hazard.

It will be appreciated that an active ingredient(s) may be added while creating the preservative composition described herein. Alternatively, the composition may be made such that the composition may be manufactured and shipped to an offsite location, such as a pharmacy, a pharmaceutical manufacturer, or cosmetic manufacturer. The composition may be added to drug products, medication, or cosmetics in an amount needed for preservation as the composition is a solution it is easy to incorporate.

The naturally occurring substance containing salicylate used in preferred embodiments is populus tremuloides bark extract which is bark of the Aspen tree. The commercial name is PhytoCide Aspen Bark Extract Powder. This particular bark is rich in salicylates, which may function as the plant's natural defense from invading parasites. The bark extract may be used in many forms, but most preferably in a powder form. The salicylates are isolated from Aspen bark prior to being used in the preservative compound described herein. Preferably, the compound containing salicylate is present in the composition in about 1 to 20% of the composition. In particular, the preferred embodiment of the composition contains about 0.1% to 5% w/w of Aspen bark extract powder.

While populus tremuloides bark extract is used in preferred embodiments, other naturally occurring substances containing salicylate may also be used in the natural preservative blend. For instance, other tree barks other than the Aspen tree also contain salicylate and may also be used. Other substances include, but are not limited to, Spiraea ulmaria L., Willow bark, Gaultheria yunnanensis, Milk Thistle (Sylibum Marianum), Betula lenta (Sweet Birch), Nettle (urtica Dioica), Betula pendula (White birch), Filipendula ulmaria (Meadowsweet), Gautheria procumbens (Wintergreen), Populus balsamifera (Balsam Poplar), Populus nigra (Black Poplar), Populus candicans (Balm Of Gilead), Salix alba (White Willow) and Viburnum prunifolium (Black Haw).

Aspen bark extract powder is typically difficult to use in compounds such as these, and its solubility can even be a deterrent. There are inherent challenges in solublizing Aspen bark extract powder, in that it takes much time, water, and heat. For this reason, it is not commonly used in compounds such as those described herein. Combining the Aspen bark extract powder with the other ingredients described herein, however, allows for an increased solubility of the extract powder. In particular, combining the other ingredients listed herein with 1 to 20% of Aspen bark extract powder allows for an increased solubility of the powder.

While there are many species of Lonicera, which is commercially known as campo plantservative WSr, Lonicera japonica and Lonicera caprifolium are utilized in preferred embodiments. Lonicera is a honeysuckle from the family Caprifoliaceae. Lonicera japonica is also referred to as Jinyinha. The preferred form of Lonicera japonica for use in the present invention is a liquid that is isolated from the plant. The contribution of Lonicera japonica to the preservative compound is to kill gram positive and gram negative bacteria. This component acts as a preservative when in liquid form. Preferably, Lonicera is present in the composition in about 1 to 20% of the composition. More specifically, the preferred embodiment of the composition contains about 0.05-1% w/w of Lonicera japonica flower extract, which may also include Lonicera caprifolium flower extract and water.

There are other alternatives to Lonicera japonica, such as the other species of Lonicera, including acuminata, aberti, albiflora, alpigena, altmannii, angustifolia, anisocalyx, arborea, arizonica, biflora, bournei, brevisepala, buchananii, buddleioides, caerulea, calcarata, calvescens, canadensis, caprifolium, carnosifolis, chrysantha, ciliosa, ciliosissima, cinerea, codonantha, confusa, conjugialis, crassifolia, cyanocarpa, dasystyla, dioica, elisae, etrusca, fargesii, ferdinandii, ferruginea, flava, fragilis, fragrantissima, fulvotomentosa, glutinosa, graebneri, gynochlamydea, hellenica, hemsleyana, heterophylla, hildebrandiana, hirsuta, hispida, hispidula, humilis, hypoglauca, hypoleuca, implexa, inconspicua, inodora, interrupta, involucrata, japonica, jilongensis, kansuensis, kawakamii, korolkowii, lanceolata, ligustrina, litangensis, longiflora, longituba, maackii, macrantha, macranthoides, maximowiczii, microphylla, minuta, minutifolia, modesta, morrowii, mucronata, myrtillus, nervosa, nigra, nitida, nubium, nummulariifolia, oblata, oblongifolia, oiwakensis, oreodoxa, orientalis, pampaninii, periclymenum, pileata, pilosa, paeflorens, prostrata, pyrenaica, reticulata, retusa, rhytidophylla, rupicola, ruprechtiana, saccata, schneideriana, semenovii, sempervirens, serreana, setifera, smilis, spinosa, splendida, standishii, stephanocarpa, subaequalis, subhispida, sublabiata, subspicata, szechuanica, taipeiensis, tangutica, tatarica, tatarinowii, tomentella, tragophylla, tricalysioides, trichogyne, trichosantha, trichosepala, tubuliflora, utahensis, villosa, virgultorum, webbiana, xylosteum, and yunnanensis.

Another component of the natural preservative blend is Leuconostoc Kimchii. Leuconostoc Kinchii is a bacillus and is one of the 15 species of Leuconostoc typically found in the Korean dietary staple Kimchii within the family of Leuconostocaceae, and is commercially known as AMS Leucidal liquid. The form most preferable is a filtrate obtained by the fermentation of the Raphanus sativus root by the microorganism leuconostoc. Leuconostoc restricts microbial growth by acidifying its environment. The most preferable species of leuconostoc for use in the present invention is leuconostoc kimchii, which is one of fifteen species of leuconostoc. Preferably, the lactic acid bacteria is present in the composition in about 0.1 to 80% w/w of the composition. In particular, the preferred embodiment of the composition contains about 10-30% leuconostoc, such as leuconostoc kimchii.

Alternative embodiments may use other species of leuconostoc, including carnosum, citreum, durionis, fallax, ficulneum, fructosum, garlicum, gasicomitatus, gelidum, inhae, lactis, mesenteroides, pseudoficulneum, and pseudomesenteroides. Further, as an alternative to leuconostoc, other lactic acid bacteria may be used, including, but not limited to, leuconostoc, including carnosum, citreum, durionis, fallax, ficulneum, fructosum, garlicum, gasicomitatus, gelidum, inhae, lactis, mesenteroides, pseudoficulneum, and pseudomesenteroides.

The fourth component of the natural preservative blend is gluconic acid. Most preferable, glucono-delta-lactone is used as the gluconic acid, which is a naturally derived product of corn fermentation. It is also a natural constituent of many foods, including honey, fruit juices, wine, and other fermented products. Glucono-delta-lactone is a salt of gluconic acid, and in an aqueous solution, there is an equilibrium between gluconic acid and the lactone. This natural food acid assists in preventing microbial growth primarily through pH depression. The multiple hydroxyl groups on the glucono-delta-lactone molecule attract water, resulting in a moisturizing effect when it is added to a formulation, such as a skin care formulation. As the pH of normal skin is around 5 to 5.5, a preferable pH of a formulation used on the skin is around 4-6, but may vary from that range depending on skin type and the specific formulation. Preferably, gluconic acid is present in the composition in about 0.05 to 10% w/w of the composition. Specifically, the preferred embodiment of the composition contains about 1-5% glucono-delta-lactone.

Other than glucono-delta-lactone, alternatives include lactobionic acid, lactic acid, mandelic acid, citric acid, glycolic acid, azelaic acid, glyceric acid, pantoic acid, ribonic acid, glucoheptonic acid, glucaric acid, glucuronic acid.

In embodiments, glycerin may be used as an additional ingredient as a vehicle. Preferably, glycerin is present in the composition in about 1 to 90% w/w of the composition. More particularly, the preferred embodiment of the composition contains about 67% glycerin. Alternatively, other components may be used in place of glycerin, such as propylene glycol, pentylene glycol, sorbitol, propanediol, propanediol dicaprylate. It will be appreciated the combination of populus tremuloides bark extract, lonicera, leuconostoc and gluconic acid increases the solubility of the populus tremuloides bark extract such that it may be used in the natural preservative blend formulation and the natural preservative blend formulation is in liquid form.

In one embodiment, the formulation contains about:
4% w/w populus tremuloides bark extract
4% w/w lonicera caprifolium and lonicera japonica
22% w/w leuconostoc kimchii
2.5% w/w gluconic acid
67.5% w/w glycerin

### EXAMPLE 1

In this example, a composition for a natural preservative blend was prepared by combining about 4% w/w populus tremuloides bark extract, 4% w/w lonicera caprifolium and lonicera japonica, 22% w/w leuconostoc kimchii, 2.5% w/w gluconic acid and 67.5% w/w glycerin. The combination was heated to 50° C. and homogenized to make a clear solution.

### EXAMPLE 2

In the second example, the natural preservative blend was prepared by combining about 4% w/w populus tremuloides bark extract, 4% w/w lonicera caprifolium and lonicera japonica, 22% w/w leuconostoc kimchii, 2.5% w/w gluconic acid and 67.5% w/w glycerin. The combination was mixed to make a clear solution.

Table 1, below, illustrates a preservative challenge test performed using about 2% of a natural preservative blend formulation described above containing populus tremuloides bark extract, lonicera caprifolium and japonica, leuconostoc kimchii, gluconic acid, and glycerin. The following formula was tested.
11% w/w Emulsifying Wax NF
5% cyclomethicone
0.4% w/w Vitamin E Acetate liquid
5% w/w Octyl Stearate
0.1% w/w EDTA disodium
2% w/w of the natural preservative blend formulation
Q.S to 100% w/w with Water

TABLE 1

| | | Day: 7 | Day: 14 | Day: 21 | Day: 28 | |
|---|---|---|---|---|---|---|
| Organisms | Inoc. Level | Dec. 21, 2008 | Dec. 28, 2008 | Jan. 04, 2009 | Jan. 11, 2009 | Pass/Fail |
| E. coli | 3.00 × 10⁶ | <10 | <10 | <10 | <10 | PASS |
| (ATCC 8739) | | | | | | |
| S. aureus | 3.75 × 10⁶ | <10 | <10 | <10 | <10 | PASS |
| (ATCC 6538) | | | | | | |
| P. aeruginosa | 3.38 × 10⁶ | <10 | <10 | <10 | <10 | PASS |
| (ATCC 9027) | | | | | | |
| C. albicans | 8.38 × 10⁶ | <10 | <10 | <10 | <10 | PASS |
| (ATCC 10231) | | | | | | |
| A. niger | 2.43 × 10⁶ | <10 | <10 | <10 | <10 | PASS |
| (ATCC 16404) | | | | | | |

The natural preservative blend formulation exhibits a broad spectrum of antimicrobial activities, such as rapid microbiocidal activity against Gram-negative bacteria, Gram-positive bacteria, yeasts, and molds. The challenge test illustrated in Table 1 above has revealed that 2% of the natural preservative blend solution in a nonionic cream base is able to successfully inhibit microbial growth. Samples were inoculated with S. aureus, E. coli, P. aeruginosa, C. albicans, and A. niger after 28 days of incubation.

Table 2 below illustrates a total log reduction of microorganisms after a 28-day Antimicrobial Effectiveness Test (AET) test in a cream. 2% of the natural preservative blend formulation+was used in the test. The composition contained populus tremuloides bark extract, lonicera caprifolium and japonica, leuconostoc kimchii, gluconic acid, and glycerin. The following formula, including the natural preservative blend formulation along with other components, was tested
11% w/w Emulsifying Wax NF
5% cyclomethicone
0.4% w/w Vitamin E Acetate liquid
5% w/w Octyl Stearate
0. 1% w/w EDTA disodium
2% w/w of the preservative solution
Q.S to 100% w/w with Water

TABLE 2

| Microorganism | Log Reduction |
|---|---|
| E. coli | 5.48 |
| S. aureus | 5.57 |
| P. aeruginosa | 5.53 |
| C. albicans | 5.92 |
| A. niger | 5.39 |

Table 3, below, illustrates the Minimum inhibitory Concentration (MIC) of the natural preservative blend formulation for 8 different microbial organisms. The following test was performed using different dilutions of the natural preservative blend containing populus tremuloides bark extract, lonicera caprifolium and japonica, leuconostoc kimchii, gluconic acid, and glycerin and water (ml of natural preservative blend formulation / ml of water). The following formula was tested:
4% w/w populus tremuloides bark extract
4% w/w lonicera caprifolium and lonicera japonica
22% w/w leuconostoc kimchii
2.5% w/w gluconic acid
67.5% w/w glycerin

TABLE 3
Natural Preservative Blend Formulation

| | 0.1 ml/ | 0.5 ml/ | 1.0 ml/ | 1.5 ml/ | 2.0 ml/ | 2.5 ml/ | 3.0 ml/ | 3.5 ml/ | 4.0 ml/ | 4.5 ml/ |
|---|---|---|---|---|---|---|---|---|---|---|
| Microorganism | 9.9 ml | 9.5 ml | 9.0 ml | 8.5 ml | 8.0 ml | 7.5 ml | 7.0 ml | 6.5 ml | 6.0 ml | 5.5 ml |
| Staphylococcus | Growth | No | No | No | No | No | No | No | No | No |
| aureus | | Growth | Growth | Growth | Growth | Growth | Growth | Growth | Growth | Growth |
| ATCC #6538 | | | | | | | | | | |
| Propionibacterium | Growth | Growth | No | No | No | No | No | No | No | No |
| acnes | | | Growth | Growth | Growth | Growth | Growth | Growth | Growth | Growth |
| ATCC #11827 | | | | | | | | | | |
| Propionibacterium | Growth | No | No | No | No | No | No | No | No | No |
| acnes | | Growth | Growth | Growth | Growth | Growth | Growth | Growth | Growth | Growth |
| ATCC #9027 | | | | | | | | | | |
| Escherichia coli | Growth | No | No | No | No | No | No | No | No | No |
| ATCC #8739 | | Growth | Growth | Growth | Growth | Growth | Growth | Growth | Growth | Growth |
| Salmonella typhi | Growth | No | No | No | No | No | No | No | No | No |
| ATCC #6539 | | Growth | Growth | Growth | Growth | Growth | Growth | Growth | Growth | Growth |
| Methicillin | Growth | No | No | No | No | No | No | No | No | No |
| Resistant | | Growth | Growth | Growth | Growth | Growth | Growth | Growth | Growth | Growth |
| Staphylococcus | | | | | | | | | | |
| aureus | | | | | | | | | | |
| ATCC #33591 | | | | | | | | | | |
| Staphylococcus | Growth | No | No | No | No | No | No | No | No | No |
| epidermidis | | Growth | Growth | Growth | Growth | Growth | Growth | Growth | Growth | Growth |
| ATCC #12228 | | | | | | | | | | |
| Aspergillus niger | Growth | No | No | No | No | No | No | No | No | No |
| ATCC #16404 | | Growth | Growth | Growth | Growth | Growth | Growth | Growth | Growth | Growth |

The present invention has been described in relation to particular embodiments, which are intended in all respects to be illustrative rather than restrictive. Since many possible embodiments may be made of the invention without departing from the scope thereof, it is to be understood that all matter herein set forth is to be interpreted as illustrative and not in a limiting sense. Alternative embodiments will become apparent to those of ordinary skill in the art to which the present invention pertains without departing from its scope.

From the foregoing, it will be seen that this invention is one well adapted to attain all the ends and objects set forth above. It will be understood that certain features and subcombinations are of utility and may be employed without reference to other features and subcombinations. This is contemplated and within the scope of the claims.

The invention claimed is:

1. A composition to be used as a natural preservative, the composition comprising a combination of:
1.0 to 20% w/w one or more naturally occurring substances containing salicylate;
0.1 to 20% w/w one or more species of lonicera;
0.1 to 80% w/w one or more leuconostoc; and
0.1 to 10% w/w gluconic acid.

2. The composition of claim 1, further comprising:
1.0 to 90% w/w glycerin.

3. The composition of claim 1, wherein the one or more naturally occurring substances containing salicylate include populus tremuloides bark extract.

4. The composition of claim 1, wherein the naturally occurring substances contain 10-60% salicylate.

5. The composition of claim 1, wherein the one or more species of lonicera include lonicera japonica.

6. The composition of claim 1, wherein the one or more lactic acid bacteria includes Leuconostoc filtrate obtained by fermentation of Raphanus sativus roots.

7. The composition of claim 6, wherein the Leuconostoc is Leuconostoc kimchii.

8. The composition of claim 1, wherein the gluconic acid is glucono-delta-lactone.

9. The composition of claim 1, wherein the composition is essentially odorless.

10. The composition of claim 1, wherein the composition is essentially colorless.

11. The composition of claim 1, further comprising:
at least one active ingredient.

12. The composition of claim 1, wherein the composition is used for the treatment of acne.

13. A composition to be used as a natural preservative, the composition comprising a combination of:
populus tremuloides bark extract;
lonicera japonica;
leuconostoc filtrate; and
glucono-delta-lactone.

14. The composition of claim 13, wherein the composition is essentially odorless and colorless.

15. The composition of claim 13, further comprising:
at least one active ingredient.

16. The composition of claim 13, wherein the composition is used as a preservative for cosmetics.

17. The composition of claim 13, wherein the composition is used as a preservative for pharmaceutical products.

18. A method for topically applying a natural preservative blend to a skin surface, the method comprising:
providing a natural preservative blend comprising one or more naturally occurring substances containing salicylate, one or more species of lonicera, one or more leuconostoc, and gluconic acid, wherein components of the natural preservative blend are provided in an amount sufficient to exhibit antimicrobial activity;
contacting the natural preservative blend with the skin surface; and
applying the natural preservative blend to the skin surface such that the natural preservative blend exhibits antimicrobial activity.

19. The method of claim 18, wherein the natural preservative blend is in liquid form.

20. The method of claim 19, wherein the natural preservative blend is miscible with organic solvents, surfactants, and emulsifiers.
